(19) United States
(12) Patent Application Publication
(10) Pub. No.: US 2018/0110538 A1
(43) Pub. Date: Apr. 26, 2018

(54) Abrasive Skin Buffer Tip

(71) Applicant: Lucinni Bailar, Houston, TX (US)

(72) Inventor: Lucinni Bailar, Houston, TX (US)

(21) Appl. No.: 15/719,077

(22) Filed: Sep. 28, 2017

Related U.S. Application Data
(60) Provisional application No. 62/412,054, filed on Oct. 24, 2016.

(51) Int. Cl. A61B 17/32; A61B 17/54; A45D 44/00

(52) U.S. Cl. CPC A61B 17/320068; A61B 2017/00761; A45D 44/00; A61B 17/54

(57) ABSTRACT

A skin buffer apparatus consists of an abrasive pad, a handle, a neck portion, and a vibrating mechanism. The vibrating mechanism provides the vibrations used for skin buffing. The vibrations are transferred onto the skin of the user via the abrasive pad. Preferably, the abrasive pad is a sandpaper pad which is used to remove an outer layer of dead skin. The handle and the neck portion allow the user to position the abrasive pad at a preferred location. The apparatus can be used on the face or any other body part where dead skin needs to be removed. The vibrating mechanism is powered through a power source. Preferably, the vibration frequency is adjustable so that different buffing processes can be completed.

The current application claims a priority to the U.S. Provisional Patent application Ser. No. 62/412,054 filed on Oct. 24, 2016.

### FIELD OF THE INVENTION

The present invention relates generally to a skin buffer device. More specifically, the present invention is an abrasive tip for a vibrating facial buffer. The rounded shape of the abrasive skin buffer tip makes it particularly conducive to applications for facial buffing. The present invention further provides a set of electronics that allows for vibration of the abrasive skin buffer tip.

### BACKGROUND OF THE INVENTION

The treatment and cleaning of skin is an area of utmost importance to modern society. Face washes, treatments, abrasive rubs, soaps, masks, brushes, and a plethora of similar items and formulas are used to remove old skin cells and dirt from the external layer of skin. The use of such items leaves the user feeling refreshed and clean, and further prevents the appearances of blemishes, such as rashes, pimples, and more. Exfoliating treatments in particular have had success in cleansing the face and body. Vibrating microdermabrasion techniques in general are optimal for facial treatment, as the increased friction from gently vibrating a rough, textured surface against the skin simultaneously improves the dirt and skin removal process when compared to manually applying soaps and friction, while avoiding the inducement of pain in the user.

However, such processes are limited by technology. While many buffing technologies improve the ability of the user to remove dirt and old skin, the tip of such cleaning tools often does not meet the necessary requirements. An optimal buffing tip cannot be too sharp or it will induce rashes, soreness, and undesirable redness in the target area. The tip cannot be too dull or it will not provide the ability to target particular areas of the face or skin. What is needed is a vibrating abrasive tip that is capable of targeting particular areas of the skin without damaging the skin area.

The present invention addresses these issues. The Abrasive Skin Buffer Tip has an abrasive surface that is capable of providing the desirable friction against the skin, without being so rough as to induce pain or discomfort. The rounded shape ensures optimal surface area for transferring vibrations from a vibrating skin buffer apparatus. The grip of the present invention allows for attachment to a variety of different vibrating skin buffer apparatuses.

### BRIEF DESCRIPTION OF THE DRAWINGS

FIG. 1 is a perspective view of the present invention.

FIG. 2 is an exploded perspective view of the present invention.

FIG. 3 is a side view of the present invention.

FIG. 4 is a block diagram illustrating the vibrating mechanism of the present invention.

### DETAIL DESCRIPTIONS OF THE INVENTION

All illustrations of the drawings are for the purpose of describing selected versions of the present invention and are not intended to limit the scope of the present invention.

The present invention is an abrasive skin buffer tip that aids in the process of buffing the skin for exfoliating purposes. More specifically, the texture of the present invention and the vibrations provided by the present invention allows the user to exfoliate skin.

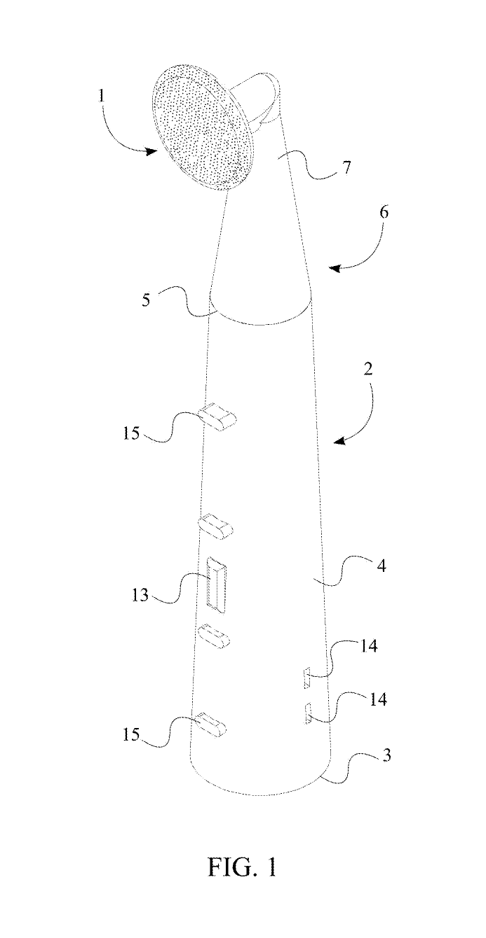

As shown in FIGS. 1-4, to execute exfoliating purposes, the present invention comprises an abrasive pad 1, a handle 2, a neck portion 6, and a vibrating mechanism 9. The abrasive pad 1 is used to be in contact with the skin and execute the exfoliating process via vibrations that are provided by the vibrating mechanism 9. The size and shape of the abrasive pad 1 can vary in different embodiments of the present invention. In the preferred embodiment, the abrasive pad 1 is a sandpaper pad which is circular in shape. The sandpaper pad is used to remove an outer layer of dead skin in a preferred area. The preferred area can be, but is not limited to, the face. Even though a sandpaper pad is used as the abrasive pad 1 in the preferred embodiment, different materials can be used in other embodiments of the present invention. The handle 2 is used to hold the present invention, while the neck portion 6, which is an extension of the handle 2, is used to position the abrasive pad 1 for exfoliating purposes per user preference.

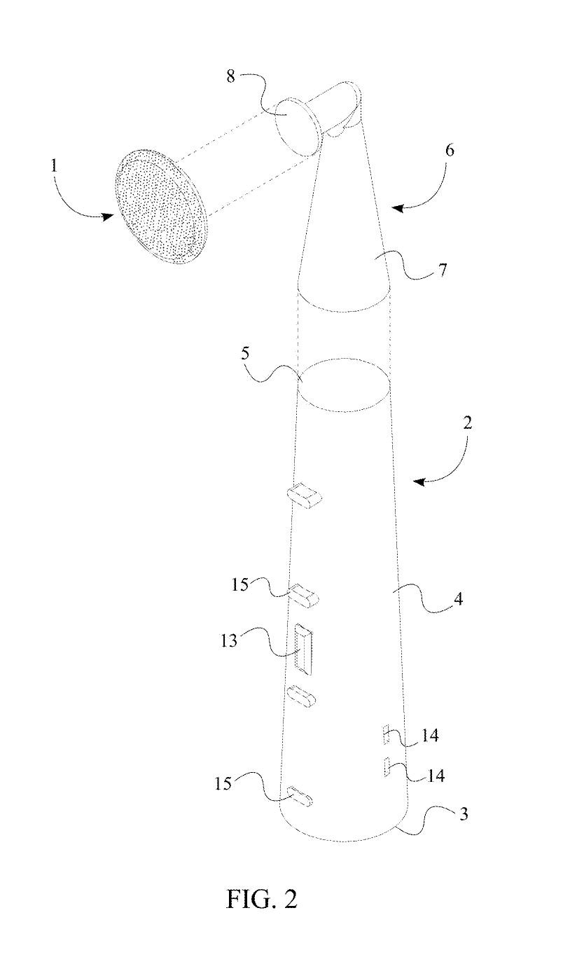

As illustrated in FIG. 2, the handle 2 comprises a first end 3, a structural body 4, and a second end 5 such that the structural body 4 extends from the first end 3 to the second end 5. The shape and size of the structural body 4 can vary in different embodiments of the present invention. Preferably, the structural body 4 is shaped to be easily gripped by the user when executing the exfoliating process. Thus, the structural body 4 has an overall cylindrical shape. In some embodiments, the structural body 4 may be tapered from the first end 3 to the second end 5. While holding the handle 2, the user can activate the vibrating mechanism 9 that is positioned within the neck portion 6 since the neck portion 6 is terminally connected to the handle 2 at the second end 5. The abrasive pad 1, which contacts the skin, is mounted onto the neck portion 6 opposite to the handle 2. In one embodiment of the present invention, the abrasive pad 1 can be removably attached to the neck portion 6. Removability of the abrasive pad 1 is beneficial if the user intends on utilizing different textures of the abrasive pad 1. In one instance, the user might prefer the abrasive pad 1 to be rough. In another instance, the user might prefer the abrasive pad 1 to be soft. In another embodiment, the abrasive pad 1 can be connected to the neck portion 6 via adhesive or another comparable method. Due to the positioning of the abrasive pad 1, when the vibrating mechanism 9 is activated, the neck portion 6 vibrates and the vibrations are transferred onto the abrasive pad 1. More specifically, the vibrating mechanism 9 is operatively coupled with the abrasive pad 1 via the neck portion 6. Ultimately, the vibrations are felt on the skin of the user via the abrasive pad 1.

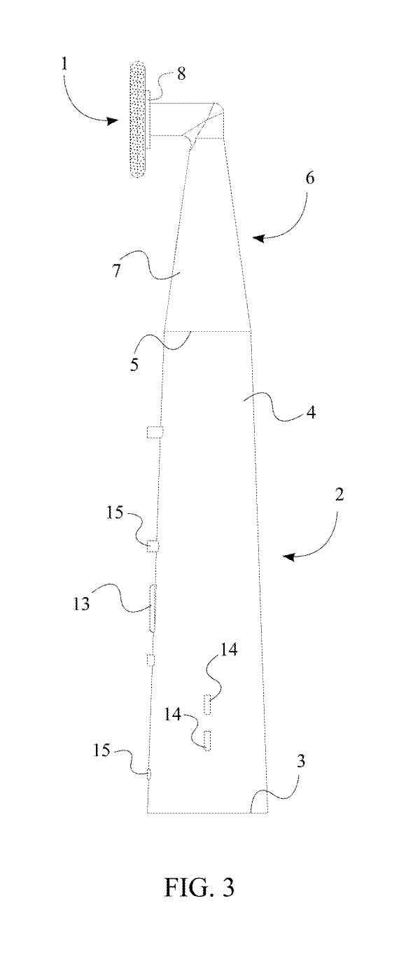

The size and shape of the neck portion 6 can vary in different embodiments of the present invention. As shown in FIG. 3, in the preferred embodiment, the neck portion 6 comprises an angled body 7 and a contact surface 8. Preferably, the neck portion 6 is made of a lightweight material such as plastic so that the material properties allows the neck portion 6 to maintain a degree of flexibility. The angled body 7 is used to orient the abrasive pad 1 to different areas on the skin. On the other hand, the contact surface 8 is used to connect and hold the abrasive pad 1. To fulfill the functionalities of the present invention, the contact surface 8 is terminally connected to the angled body 7 opposite to the handle 2. To accommodate the circular shape of the abrasive pad 1, the contact surface 8 is also circular in shape in the preferred embodiment of the present invention. However, the size and shape of the contact surface 8 can vary in different embodiments of the present invention.

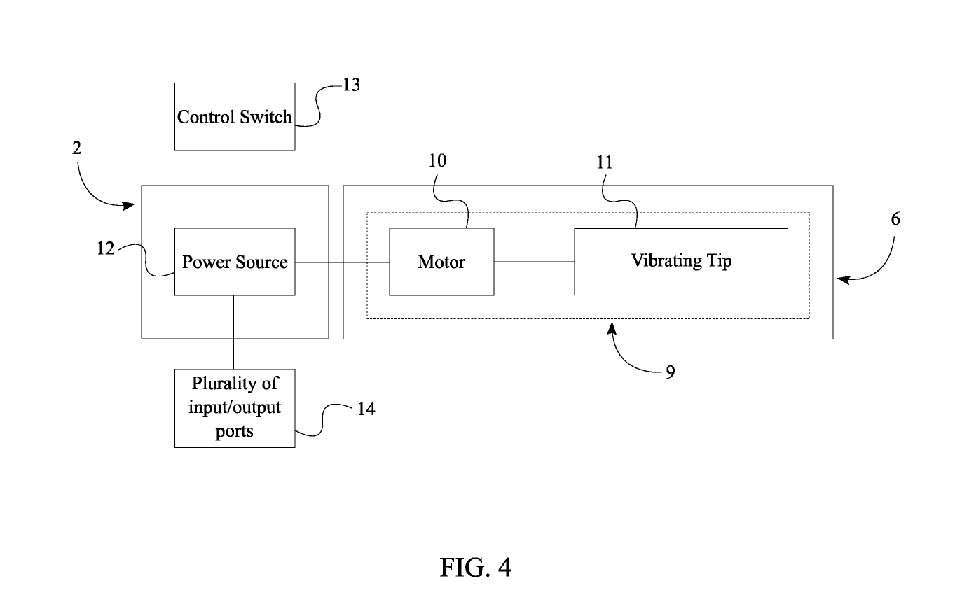

The present invention further comprises a power source 12 that is used for powering the vibrating mechanism 9. In the preferred embodiment, the power source 12 is a rechargeable battery. However, the power source 12 can vary in different embodiments of the present invention. For example, in some embodiments the power source 12 may be a power cord that can be plugged into a wall outlet or power adapter. To provide the necessary power to the vibrating mechanism 9, the power source 12 is electrically connected to the vibrating mechanism 9 as illustrated in FIG. 4. In the preferred embodiment of the present invention, the vibrating mechanism 9 comprises a motor 10 and a vibrating tip 11. The motor 10 receives electrical power by being electrically connected to the power source 12. Next, the motor 10 activates the vibrating tip 11 by being mechanically engaged with the vibrating tip 11. To position the power source 12 distant from the abrasive pad 1, and to maintain an aesthetically pleasing appearance, the power source 12 is positioned within the structural body 4.

To activate and deactivate the vibrating mechanism 9, the present invention further comprises a control switch 13 that is mounted onto the structural body 4. The positioning of the control switch 13 allows the user to promptly activate or deactivate the vibrating mechanism 9 and hence, the vibrations applied to the skin. To do so, the control switch 13 is electronically connected to the vibrating mechanism 9. Additionally, the control switch 13 can be configured to control the vibration frequency of the vibrating mechanism 9. Different frequencies can be beneficial to exfoliate skin in different areas of the body. As an example, a higher vibration frequency can be used to exfoliate skin on the foot. On the other hand, a lower vibration frequency can be used to exfoliate skin on the face.

As previously discussed, the power source 12 can be, but is not limited to, a rechargeable battery. To recharge the power source, the present invention further comprises at least one input port 14 that traverses into the structural body 4. To execute charging functionalities, the at least one input port 14 is electronically connected to the power source 12.

When utilizing the present invention, maintaining a firm grip is essential. To aid with the process, the present invention further comprises a plurality of gripping portions 15 that is externally distributed along the structural body 4 in between the first end 3 and the second end 5. The rough texture of the plurality of gripping portions 15 provides a non-slip holding area for the user.

The following process flow is generally followed when utilizing the present invention. Initially, the user holds the present invention by the handle 2 and orients the neck portion 6 so that the abrasive pad 1 is at the preferred area on the skin. When the abrasive pad 1 is positioned as preferred, the user activates the vibrating mechanism 9 via the control switch 13. The frequency of the vibrations can also be adjusted via the control switch 13. In the preferred embodiment of the present invention, 30,000-40,000 vibrations are produced per minute. Next, by pressing the abrasive pad 1, which is a sandpaper pad in the preferred embodiment, the exfoliating process can be executed. If the abrasive pad 1 is removable, the abrasive pad 1 can be replaced according to user preference. If the power source 12 needs to be recharged, the user utilizes the at least one input port 14 to recharge the power source 12. By utilizing the present invention, wrinkles can be reduced, pores can be minimized, acne scars can be reduced, elasticity of the skin can be improved, stretch marks can be reduced, and uneven skin tones can be improved,

Although the invention has been explained in relation to its preferred embodiment, it is to be understood that many other possible modifications and variations can be made without departing from the spirit and scope of the invention as hereinafter claimed.

What is claimed is:

1. An abrasive skin buffer tip comprises:
an abrasive pad;
a handle;
a neck portion;
a vibrating mechanism;
the handle comprises a first end, a structural body, and a second end;
the structural body extending from the first end to the second end;
the vibrating mechanism being positioned within the neck portion;
the neck portion being terminally connected to the handle at the second end; and
the abrasive pad being mounted onto the neck portion opposite to the handle, wherein the vibrating mechanism is operatively coupled with the abrasive pad via the neck portion.

2. The abrasive skin buffer tip as claimed in claim 1, wherein the abrasive pad is a sandpaper pad.

3. The abrasive skin buffer tip as claimed in claim 1 further comprises:
the neck portion comprises an angled body and a contact surface; and
the contact surface being terminally connected to the angled body opposite to the handle.

4. The abrasive skin buffer tip as claimed in claim 1, wherein the contact surface is circular in shape.

5. The abrasive skin buffer tip as claimed in claim 1, wherein the abrasive pad is circular in shape.

6. The abrasive skin buffer tip as claimed in claim 1 further comprises:
a power source;
the power source being positioned within the structural body; and
the power source being electrically connected to the vibrating mechanism.

7. The abrasive skin buffer tip as claimed in claim 6, wherein the power source is a rechargeable battery.

8. The abrasive skin buffer tip as claimed in claim 1 further comprises:
the vibrating mechanism comprises a motor and a vibrating tip;
the motor being electrically connected to a power source; and
the motor being mechanically engaged with the vibrating tip.

9. The abrasive skin buffer tip as claimed in claim 1 further comprises:
a control switch;
the control switch being mounted onto the structural body; and
the control switch being electronically connected to the vibrating mechanism.

10. The abrasive skin buffer tip as claimed in claim 1 further comprises:
at least one input port;
the at least one input port traversing into the structural body; and
the at least one input port being electronically connected to the power source.

11. The abrasive skin buffer tip as claimed in claim 1 further comprises:
a plurality of gripping portions; and
the plurality of gripping portions being externally distributed along the structural body between the first end and the second end.

12. The abrasive skin buffer tip as claimed in claim 1, wherein the abrasive pad is removably attached to the neck portion opposite to the handle.

13. The abrasive skin buffer tip as claimed in claim 1, wherein the abrasive pad is connected to the neck portion opposite to the handle.
